(19) United States
(12) Patent Application Publication
(10) Pub. No.: US 2026/0056216 A1
(43) Pub. Date: Feb. 26, 2026

(54) ACUTE CORTICOID STRESS INDEX IN FISH

(71) Applicant: Innolipid AS, Ålesund (NO)

(72) Inventors: Asgeir Sæbø, Ålesund (NO); Ernestine Fanjara, Ålesund (NO); Vera Kristinova, Ålesund (NO)

(21) Appl. No.: 19/103,036

(22) PCT Filed: Aug. 10, 2023
(86) PCT No.: PCT/IB2023/058083

§ 371 Date: Feb. 11, 2025

Related U.S. Application Data
(60) Provisional application No. 63/397,543, filed on Aug. 12, 2022.

(51) Int. Cl. G01N 33/74; A01K 61/10

(52) U.S. Cl. CPC G01N 33/743; A01K 61/10; G01N 2333/4603; G01N 2800/7004

(57) ABSTRACT

The present innovation provides a tool for measuring acute and long-term stress in farmed fish, based on non-invasive sampling of material for analysis, wherein derivatives of stress hormones are quantified. The material analysed comprises a sample (e.g., feces samples, water samples from cages or tanks holding the farmed fish, and samples collected from filtration of water from cages or tanks holding the farmed fish) collected at any stage during the life cycle as well as scales, fins, blood, mucus, and bile obtained upon slaughtering. The analyte are specific corticoid metabolites and conjugates.

### CROSS-REFERENCE TO RELATED APPLICATIONS

This application claims the benefit of U.S. Provisional Application No. 63/397,543, filed Aug. 12, 2022, the contents of which are herein incorporated by reference in their entirety.

### FIELD OF THE INVENTION

The present innovation provides a tool for measuring acute and long-term stress in farmed fish, based on non-invasive sampling of material for analysis, wherein derivatives of stress hormones are quantified.

### BACKGROUND OF THE INVENTION

Intensive livestock production follows a business model based on exploding economics of scale, with the main objective to maximize profitability. The driver in this trend is partly financial institutions investing billions of dollars in the world's biggest companies in chicken, pork and salmon industry and may contribute to animal welfare infringement on a massive scale.

That welfare in fish farming has not been properly addressed is observed from various problems like rate of survival, necessity of frequent delousing operations, various virus disease problems, and sudden cardiac deaths. Sub optimal welfare represent a loss to the farming industry and a concern to the authorities and also retail customers. Stress alleviation has for a long time been anticipated a superior approach for improved welfare in fish farming. Progress in this field, however, has been halted due to lack of tools for measuring stress in fish, and in particular to obtain samples in a non-invasive way. This invention provides a tool for accurate quantification of both acute and long-term stress in fish farming

### SUMMARY OF THE INVENTION

The level of cortisol in blood is a well-known marker of stress in a number of animal species as well as in humans. However, to obtain a sample of blood for analysis of cortisol is not feasible. It is well known that cortisol also can be found in feces and that this reflects the stress level the previous day. A few years ago, it was proposed to use level of cortisol in feces as a measure of stress level. Cortisol was quantified in a diluted sample of feces by Enzyme Linked Immunosorbent Assay (ELISA).

The present innovation however is based on quantification of more than two corticoid metabolites wherein, for example, free cortisol comprises about 1% of the analytes, and conjugated forms of cortisol comprise about 20% of the analytes. The analytes included were discovered by searching for cortisol metabolites in stressed salmon, screening for 24 potential metabolites, each occurring potentially in at least three different forms, either free or in two conjugated forms. In some preferred embodiments, including all the discovered 6 metabolites/conjugated forms greatly improved the robustness and accuracy of the quantification of stress level in fish and allowed for defining a corticoid stress index based on the metabolites. The index, named Acute Corticoid Stress Index, ACSI, is defined as sum of the selected corticoid metabolites and usually reported as nanograms (ng)/gram feces. The technology may also provide a long-term stress level index reported as Cumulative Acute Corticoid Stress Index, CACSI, wherein data represents an index based on pooling samples over time (one week or more) and further pooling multiple datasets representing months and years.

Accordingly, in one aspect, the present invention provides a method of providing a stress index for farmed fish comprising calculating an acute corticoid stress index (ACSI) for farmed fish expressed as the sum of: 1) two or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone; 2) two or more of cortisol, cortisone, 5b-dihydrocortisone, and 5b-tetrahydrocortisone; or 3) the sum 5b-dihydrocortisone in free or conjugated form and 5b-tetrahydrocortisone in free or conjugated form; in a sample selected from the group consisting of feces samples, water samples from cages or tanks holding the farmed fish, and samples collected from filtration of water from cages or tanks holding the farmed fish.

In another aspect, the present invention provides methods of assessing stress in farmed fish comprising: calculating an acute corticoid stress index (ACSI) for farmed fish expressed as the sum of: 1) two or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone; 2) two or more of cortisol, cortisone, 5b-dihydrocortisone, and 5b-tetrahydrocortisone; or 3) the sum 5b-dihydrocortisone in free or conjugated form and 5b-tetrahydrocortisone in free or conjugated form; in a sample selected from the group consisting of feces samples, water samples from cages or tanks holding the farmed fish, and samples collected from filtration of water from cages or tanks holding the farmed fish and comparing the calculated ACSI to a reference or control level, wherein an elevated ACSI as compared to the reference or control level is indicative of stress.

In some preferred embodiments, the ACSI is the sum of three or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample. In some preferred embodiments, the ACSI is the sum of four or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample. In some preferred embodiments, the ACSI is the sum of five or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample. In some preferred embodiments, the ACSI is the sum of six or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample. In some preferred embodiments, the ACSI is the sum of seven or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample. In some preferred embodiments, the ACSI is the sum of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample.

In some preferred embodiments, ACSI is the sum of cortisol, cortisone, 5b-dihydrocortisone, and 5b-tetrahydrocortisone in the sample. In some preferred embodiments, ACSI consists of or consists essentially of sum of cortisol, cortisone, 5b-dihydrocortisone, and 5b-tetrahydrocortisone in the sample.

In some preferred embodiments, the ASCI comprises the sum 5b-dihydrocortisone in free or conjugated form and 5b-tetrahydrocortisone in free or conjugated form. In some preferred embodiments, the ACSI further comprises the sum of cortisol in free or conjugated form and/or cortisone in free or conjugated form in addition to the sum of 5b-dihydrocortisone in free or conjugated form and 5b-tetrahydrocortisone in free or conjugated form. In some preferred embodiments, the ASCI consists of or consists essentially of the sum 5b-dihydrocortisone in free or conjugated form and 5b-tetrahydrocortisone in free or conjugated form. In some preferred embodiments, ASCI consists of or consists essentially of the sum of cortisol in free or conjugated form and/or cortisone in free or conjugated form in addition to the sum of 5b-dihydrocortisone in free or conjugated form and 5b-tetrahydrocortisone in free or conjugated form.

In some preferred embodiments, the ACSI is expressed as ng/gram feces.

In some preferred embodiments, the ACSI is a Cumulative ACSI (CACSI) calculated from a) the pooled sample analyzed represents a group of more than 10 individuals, b) the samples are collected at 5 or more time slots or continuously for subsequent pooling of data c) the sample material analyzed is a sample selected from the group consisting of feces samples, water samples from cages or tanks holding the farmed fish, and samples collected from filtration of water from cages or tanks holding the farmed fish and d) the analyte quantified comprises one or more or, or preferably to or more of, cortisol, cortisone, 5b-dihydrocortisone, and 5b-tetrahydrocortisone.

In some preferred embodiments, the farmed fish are a salmonid. In some preferred embodiments, the salmonid is Salmo salar.

In some preferred embodiments, the farmed fish are a population of farmed fish.

In some preferred embodiments, the sample is a feces sample.

In some preferred embodiments, the sample is a water sample from cages or tanks holding the farmed fish.

In some preferred embodiments, the sample is a sample collected from filtration of water from cages or tanks holding the farmed fish.

In some preferred embodiments, the present invention provides an ACSI produced according to any of the foregoing methods.

In some preferred embodiments, the present invention provides an acute corticoid stress index (ACSI) for farmed fish expressed as the sum of two or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in a sample selected from the group consisting of feces samples, water samples from cages or tanks holding farmed fish, and samples collected from filtration of water from cages or tanks holding farmed fish. In some preferred embodiments, the ACSI is the sum of three or more, four or more, five or more, six or more, seven or more or all eight of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in a sample selected from the group consisting of feces samples, water samples from cages or tanks holding farmed fish, and samples collected from filtration of water from cages or tanks holding farmed fish.

In some preferred embodiments, the present invention provides a cumulative acute corticoid stress index (ACSI) for farmed fish calculated from multiple measurements of one or several acute stress markers, wherein a) the pooled sample analyzed represents a group of more than 10 individuals, b) the samples are collected at 5 or more time slots or continuously for subsequent pooling of data c) the sample material analyzed is a sample selected from the group consisting of feces samples, water samples from cages or tanks holding farmed fish, and samples collected from filtration of water from cages or tanks holding farmed fish, and d) the analyte quantified is any of cortisol, cortisone, 5b-dihydrocortisone, and 5b-tetrahydrocortisone.

In some preferred embodiments, data obtained from analysis of any of bile, mucus, fins, scale, and blood is also included in the index. In some preferred embodiments, the index further comprises batch record data on fish handling including but not limited to delousing, vaccination, relocation, smoltification and disease outbreak.

In some preferred embodiments, the ACSI is provides as information or quality of product information or as a part of a product certificate upon trading of fish.

In some preferred embodiments, the ACSI is provided to retail customers as product quality information.

In some preferred embodiments, the present invention provides methods of providing a customer a value or a number or a set of numbers established from one or several algorithms based on analytical data obtained according to the methods described above under the tag “Acute Corticoid Stress Index” ACSI, or “Cumulative Acute Corticoid Stress Index” CACSI, or Chronic Corticoid Stress Index” CCSI” or simply “Stress index”.

In some preferred embodiments, the number or a part of the number expressing the stress level is quantified as relative to an empirical maximum or minimum level of a said particular analyte or analytes.

In some preferred embodiments, the present invention provides methods of providing a corticoid stress index for a fish or a population of fish or a product made from fish according to the methods described above wherein the corticoid analyte quantified comprises any of cortisol, cortisone, glucuronidated cortisol, sulphated cortisol, glucuronidated cortisone, sulphated cortisone, glucuronidated 5b-di hydrocortisone, sulphated 5b-di hydrocortisone, glucuronidated 5b-tetra hydrocortisone, sulphated 5b-tetra hydrocortisone, sulphated 5b-dihydrocortisol, sulphated 5b-dihydrocortisol, glucuronidated 5b-tetrahydrocortisol, sulphated 5b-tetrahydrocortisol, glucuronidated b-cortolone, and sulphated b-cortolone.

In some preferred embodiments, the present invention provides methods of providing data on stress in a farmed fish comprising quantifying the level of any of, glucuronidated 5b-di hydrocortisone, sulphated 5b-di hydrocortisone, glucuronidated 5b-tetra hydrocortisone, sulphated 5b-tetra hydrocortisone, sulphated 5b-dihydrocortisol, sulphated 5b-dihydrocortisol, glucuronidated 5b-tetrahydrocortisol, sulphated 5b-tetrahydrocortisol, glucuronidated b-cortolone, and sulphated b-cortolone, alone or in any combination or in any relative quantities within any grouping of said analytes.

In some preferred embodiments, the present invention provides methods of quantifying a stress response in fish comprising determined the amount of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in feces of salmon.

In some preferred embodiments, the present invention provides a quality-of-life certificate for a fish including both a Stress Index value and an Omega-3 index based on analysis of blood obtained from the same group of fish, for instance during the slaughtering process.

In some preferred embodiments, the present invention provides methods comprising providing ACSI information described as above or made by a method as described above with a fish product offered for sale to retail customers wherein the information is either provided in print on a label of said fish product or obtained via an app scanning a label on the product.

### DETAILED DESCRIPTION OF THE INVENTION

Stress is a major issue in fish farming accounting for substantial loss and suboptimal animal welfare. Stress has an impact on feeding behavior, growth, and the immune system. Substantial knowledge has been generated over the years on how to reduce stress in fish populations, even though hitherto there has been a lack of reliable tools for measuring stress in fish.

The problem solved by the instant invention is the lack of a tool for measuring stress in fish experienced by the fish itself. Well understood and described markers of stress are readily available in plasma of fish but cannot be used on a regular basis for stress measurements since obtaining blood samples are very stressful to the fish. Moreover, in addition to single point measurements of stress, there also is an unmet need for quantification of long-term stress in fish farming. The problem has been addressed by looking at deposition of stress markers in the scale and fins of fish. However, unless the fish is sacrificed, sampling will impose great stress to the fish. Moreover, fish welfare is poorly reflected by cortisol content in scale since the character and duration of stress cannot be understood from a final accumulated level of the stress marker. It is also well known that plasma cortisol level and hence level deposited in scale show high individual variation.

Recent attempts to quantify stress hormones obtained by a non-invasive method comprise quantification of cortisol in the water and/or in feces. A monitoring system analyzing the water may be a viable approach, but there are some major hurdles to quantify cortisol levels in a dynamic water circulation system in a meaningful way. Although the approach could work in a specific farming setup, the data obtained would be hard to compare to another setup. Measuring corticoids in the water is not feasible in open pens in the sea.

The levels of cortisol and cortisone have been determined in a number of species, including several fish species. It has further been confirmed in intervention studies that those markers increase upon induced stress. However, stress quantified as level of corticoids in feces has several problems. First, the data obtained is only reflecting acute stress about a day before collection and then leveling off. Second, several stressful actions (relocation, delousing, vaccination etc.) are usually undertaken after a period of starvation and hence there will be little or no feces to sample for analysis. Moreover, which analyte precisely to be quantified in fish feces of farmed fish such as salmonids has not been reported.

ELISA has been used as a method to quantify cortisol in feces⁽¹⁾, but the data obtained may well be far out of range since several corticoid-like analytes interfere with the cortisol.

Whereas cortisol is the major stress corticoid markers rapidly increasing in blood as a response to acute stress in fish, studies conducted on rainbow trout showed that it is quickly converted into cortisone after the onset of stress⁽²⁾. Cortisol and cortisone are then further metabolized in the liver into various derivatives in the form of glucuronidated and sulphated metabolites. These comprise the major potential analytes that can be quantified in bile and/or feces of the fish.

Recently Meling et. al.⁽³⁾ developed a method wherein cortisol was accurately determined after decoupling the glucuronidated and sulphated conjugates of cortisol followed by Liquid Chromatography coupled with Tandem Mass Spectrometry quantification. However, the present inventors found that cortisol (free, glucuronidated and sulphated together) comprised only about 20% of corticoid metabolites in the feces of stressed salmon. As for cortisol, the present inventors found that about 82% was in sulphated form, 15% in glucuronidated form and only about 3% was in free form. Further, the present inventors also observed that the decline in various corticoid metabolites in feces do not follow the same time pattern as cortisol. Therefore, the “wave” of cortisol over time in faces will be in front of the wave of various other metabolites derived from cortisol and level off at a later point of time. Therefore, the acute corticoid stress index provided in this invention is based on the sum of the waves represented by the various corticoid analytes disclosed herein.

As for the analyte to be quantified in the present innovation, the approach described in the instant invention is to look for the actual metabolites/conjugates present and, in particularly preferred embodiments, to look for corticoid analytes in samples from stressed fish. Based on literature on corticoid metabolism in various species and humans, 24 different metabolite standards were obtained and analyzed to determine which metabolite and in which conjugated form the metabolites were these were present in salmon.

The metabolites and conjugates were determined by HPLC coupled to tandem mass spectrometry (QTOF). The analytes quantified in feces were cortisol, cortisone, glucuronidated cortisol, sulphated cortisol, glucuronidated cortisone, sulphated cortisone, glucuronidated 5β-dihydrocortisone, sulphated 5β-dihydrocortisone, glucuronidated 5β-tetrahydrocortisone, sulphated 5β-tetrahydrocortisone, glucuronidated 5β-tetrahydrocortisol, sulphated 5β-tetrahydrocortisol, glucuronidated β-cortolone, and sulphated β-cortolone.

Surprisingly, it was found that cortisol and its conjugates comprised only about 20% of the useful markers of stress. This discovery offered a great opportunity for the cleanup of the analyte and the overall purity and robustness of an Acute Corticoid Stress Index (ACSI). First, less material is needed for sample preparation which practically means that the sample can be diluted about 10 to 20 times more. Second, a cleaner sample results in more efficient enzymatic reactions, more complete removal of the conjugates and less impurities added to the column and detector. In some particularly preferred embodiments, the ACSI is most accurately determined as the sum of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone, determined after enzymatic decoupling.

It is contemplated that ACSI may provide accurate and valuable information about a stress response to various incidences. However, data on acute response to particular stress situations may be of limited value as a tool for defining general animal welfare.

Having established a robust and validated method to determine ACSI, the present inventors set out to develop a method to provide a long-term stress index in fish. This method preferably covers an entire period in the life cycle, including an all over lifetime stress index. To be useful, this tool should be attractive for farmers, consumers, and authorities as well, for improving fish welfare.

Accordingly, in some preferred embodiments, the present invention provides methods to provide a chronic stress index, (Cumulative Acute Corticoid Stress Index), CACSI, based on pooling of ACSI data. In one embodiment of the invention, the aim is to provide CACSI expressing the stress level experienced by the herd the fish belongs to, and further representing a certain period of life of said herd. This may apply to a specific period of development of the reared batch, for instance from fingerlings to fully developed smolt in salmon. Samples of feces, or faecal material automatically removed or filtered from the tank, may be pooled every week for analysis and will thus represent the average level of stress markers from a group of fish over a defined time window. In this way the problem with individual variation, diurnal variation, and the lack of chronic character of ACSI are overcome. Any major incidence of questionable welfare protocol or any accident imposing stress over the period will be reflected in an overall enhanced level of the markers in one or several of the pooled samples. Even if the pooled samples do not cover a time window completely, regular samplings will provide a CACSI of great value for judgement of stress level experienced by the members of the fish population.

In one embodiment of this invention, CACSI on a batch of smolt ready for shipment to a fish farmer, can be provided as a quality parameter.

In another embodiment of this invention, a fish farmer may decide to increase the frequency of data collection and thereby closely monitor for potential outbreak of a virus disease or a blooming of toxic algae. The ACSI of the selected period will be more or less the same, despite including more data points to be able to address a disease outbreak or other problems.

In yet another embodiment of the invention, data included in a CACSI may also comprise analytical data on corticoids in bile, gut mucus, blood, skin mucus and/or scale from material obtained in the slaughtering process. Fish feces is not always obtainable since fish feeding is often terminated prior to major treatments like delousing, relocation, and slaughtering. Therefore, to strengthen the accuracy and value of the CACSI, analytical data obtained from material collected postmortem may be included in the dataset. In particular, a CACSI of a slaughtered batch may include stress markers quantified in gut mucus, bile (representing stress level a couple of days up to sacrifice) and in blood. The latter would be verifying that the killing procedure worked as intended confirming that the fish was unconscious at the moment of sacrifice.

In yet another embodiment of the invention, the algorithm for providing the CACSI may also incorporate batch data records on all major batch operations supposed to induce stress. This includes but are not limited to delousing, vaccination, and relocation. While gut mucus may be obtainable after such operations are performed, data from feces will not be obtainable since a period starvation usually is performed on beforehand.

It should at this point be clear that a CACSI can be provided based on a dataset/algorithm from multiple analysis of faecal samples covering long periods and even the entire life cycle of a salmon from the hatchery to the slaughterhouse. Fully implemented, this tool will become a valuable tool for fish farmers, traders, retail customers and authorities alike.

### EXPERIMENTAL

### Example 1

A group of 40 Atlantic salmon smolt weighing between 100 and 500 g, ready for sea transfer were used for the experiment. Twenty fish were sampled, euthanized, and stripped for feces and 20 fish subject to moderate handling and acute hypoxia by briefly removing the fish from water. The latter were sampled, euthanized, and stripped for feces 24 h after the onset of the stress event. Feces sampled before and after the stress event were pooled into one sample respectively and designated as Control and Stress group. Fish were euthanized with Benzoak at a concentration of 0.6 mL/L before feces sampling in accordance with the Norwegian regulations on animal welfare.

Analytical standards of 24 different potential metabolites of cortisol (known from other species and human) were purchased and used as reference in the search for corticoid metabolites in pooled samples of feces from the stressed group using UHPLC coupled to Tandem Mass Spectrometry (QTOF) for analysis. The column used was an ACQUITY UPLC BEH C18 Column (130 A, 1.7 μm, 2.1 mm×75 mm).

Major metabolites/conjugates detected was then quantified also in the control group. The data obtained is summarized in table below.

| | |
|---|---|
| Control | Stressed |
| group | Group |

| | |
|---|---|
| Corticoid metabolite/conjugate | ng/gram wet feces |

| | | |
|---|---|---|
| Cortisol | 7.92 | 8.47 |
| Glucuronidated cortisol | 33.81 | 85.49 |
| Sulphated cortisol | 128.11 | 742.16 |
| Cortisone | <LOD | <LOD |
| Glucuronidated cortisone | 6.55 | 13.29 |
| Sulphated cortisone | 199.63 | 892.60 |
| 5b-dihydrocortisone | <LOD | <LOD |
| Glucuronidated 5b-dihydrocortisone | 33.38 | 136.19 |
| Sulphated 5b-dihydrocortisone | 55.95 | 344.82 |
| 5b-tetrahydrocortisone | <LOD | <LOD |
| Glucuronidated 5b-tetrahydrocortisone | 178.41 | 691.32 |
| Sulphated 5b-tetrahydrocortisone | 107.74 | 772.27 |

### Example 2

Samples of feces were collected continuously by a device removing solids from the rearing tank, pooled weekly for analysis until the size of the fish was about 140 grams, and the fish were ready for smoltification. Data were pooled to represent monthly periods. After transfer of the batch to sea pen, collection of feces was continued on a weekly basis from more than 10 individuals and weekly data were pooled to represent monthly averages. When the salmon batch was fully grown and slaughtered, monthly ACSI from the entire life cycle (26) were pooled to obtain a Cumulative ACSI 1530 ng/g.

### Example 3

Bile was collected using a syringe with needle from euthanized and dissected fish. Like feces, the bile sample was diluted, subject to enzymatic and acid hydrolysis and analyzed as in example 1. The corticoid metabolites detected in feces (example 1) were also detected, not unexpectedly, in the bile in a more concentrated form. Bile readily available in the slaughtering process, is therefore a matrix useful for acute stress analysis providing data on stress prior to the slaughtering process (when feces is not available). Data from bile and also data from blood samples and scale, may therefore be added to a life Acute stress index (LACSI), to provide proof of good animal welfare connected to dislocation and slaughtering.

### CITED LITERATURE

1) Yanran Cao et. al. Establishment of a non-invasive method for stress evaluation in farmed salmon based on direct fecal corticoid metabolites measurement. Fish & Shellfish immunology 66 (2017) 317-324
2) Pottinger, T. G. and T. A. Moran, Differences in plasma cortisol and cortisone dynamics during stress in two strains of rainbow trout (Oncorhynchus mykiss). Journal of Fish Biology, 1993. 43(1): p. 121-130.
3) Cato Brede et. al.: Monitoring Farmed Fish Welfare by Measurement of Cortisol as a stress Marker in Fish Feces by Liquid Chromatography Coupled with Tandem Mass Spectrometry. Molecules 2022, 27, 2481

1. A method of providing a stress index for farmed fish comprising calculating an acute corticoid stress index (ACSI) for farmed fish expressed as the sum of: 1) two or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone; 2) two or more of cortisol, cortisone, 5b-dihydrocortisone, and 5b-tetrahydrocortisone; or 3) the sum 5b-dihydrocortisone in free or conjugated form and 5b-tetrahydrocortisone in free or conjugated form; in a sample selected from the group consisting of feces samples, water samples from cages or tanks holding the farmed fish, and samples collected from filtration of water from cages or tanks holding the farmed fish.

2. The method of claim 1, further comprising:
comparing the calculated ACSI to a reference or control level, wherein an elevated ACSI as compared to the reference or control level is indicative of stress.

3. The method of claim 1, wherein the ACSI is the sum of three or more of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample.

4.-7. (canceled)

8. The method of claim 1, wherein the ACSI is the sum of glucuronidated cortisol, sulphated cortisol, glucuronidated cortisone, sulphated cortisone, glucuronidated 5b-dihydrocortisone, sulphated 5b-dihydrocortisone, glucuronidated 5b-tetrahydrocortisone, and sulphated 5b-tetrahydrocortisone in the sample.

9. The method of claim 1, wherein the ACSI is the sum of cortisol, cortisone, 5b-dihydrocortisone, and 5b-tetrahydrocortisone, in the sample.

10. The method of claim 1, wherein the ASCI comprises the sum 5b-dihydrocortisone in free or conjugated form and 5b-tetrahydrocortisone in free or conjugated form.

11. The method of claim 10, wherein the ACSI further comprises the sum of cortisol in free or conjugated form and/or cortisone in free or conjugated form in addition to the sum of 5b-dihydrocortisone in free or conjugated form and 5b-tetrahydrocortisone in free or conjugated form.

12. (canceled)

13. The method of claim 1, wherein the ACSI is a Cumulative ACSI (CACSI) calculated from a) the pooled sample analyzed represents a group of more than 10 individuals, b) the samples are collected at 5 or more time slots or continuously for subsequent pooling of data c) the sample material analyzed is in a sample selected from the group consisting of feces samples, water samples from cages or tanks holding the farmed fish, and samples collected from filtration of water from cages or tanks holding the farmed fish, and d) the analyte quantified comprises two or more of cortisol, cortisone, 5b-dihydrocortisone, and 5b-tetrahydrocortisone.

14. The method of claim 1, wherein the farmed fish is/are a salmonid.

15. The method of claim 14, wherein the salmonid is Salmo salar.

16. The method of claim 1, wherein the farmed fish are a population of farmed fish.

17. The method of claim 1, wherein the

18. The method of claim 1, wherein the sample is a water sample from cages or tanks holding the farmed fish.

19. The method of claim 1, wherein the sample is a sample collected from filtration of water from cages or tanks holding the farmed fish.

20. (canceled)

21. (canceled)

22. (canceled)

23. A cumulative acute corticoid stress index (CACSI) for farmed fish calculated from multiple measurements of one or several acute stress markers, wherein a) the pooled sample analyzed represents a group of more than 10 individuals, b) the samples are collected at 5 or more time slots or continuously for subsequent pooling of data c) the sample material analyzed is a sample selected from the group consisting of feces samples, water samples from cages or tanks holding farmed fish, and samples collected from filtration of water from cages or tanks holding farmed fish, and d) the analyte quantified comprises one or more of cortisol, cortisone, 5b-dihydrocortisone, and 5b-tetrahydrocortisone.

24. A cumulative acute corticoid stress index according to claim 23, wherein data obtained from analysis of any of bile, mucus, fins, scale, and blood is also included.

25. A cumulative acute corticoid stress index according to claim 23 further comprising batch record data on fish handling including but not limited to delousing, vaccination, relocation, smoltification and disease outbreak.

26. A cumulative acute corticoid stress index according to claim 23 provided as information or quality of product information or as a part of a product certificate upon trading of farmed fish.

27. A cumulative acute corticoid stress index according to claim 23 provided to retail customers as product quality information.

28. (canceled)

29. A method of providing a stress index according to claim 1 wherein the number or a part of the number expressing the stress level is quantified as relative to an empirical maximum or minimum level of a said particular analyte or analytes.

30.-34. (canceled)
